(19) United States
(12) Patent Application Publication
(10) Pub. No.: US 2023/0363710 A1
(43) Pub. Date: Nov. 16, 2023

(54) WEARABLE SENSOR AND WEARABLE SENSOR SYSTEM

(71) Applicant: Hitachi, Ltd., Tokyo (JP)

(72) Inventors: Yu YAMADA, Tokyo (JP); Hiroyuki YOSHIMOTO, Tokyo (JP)

(73) Assignee: Hitachi, Ltd., Tokyo (JP)

(21) Appl. No.: 18/313,541

(22) Filed: May 8, 2023

(30) Foreign Application Priority Data
May 12, 2022 (JP) 2022-078857

(51) Int. Cl. A61B 5/00

(52) U.S. Cl. CPC A61B 5/6806

(57) ABSTRACT

A wearable sensor that is worn on a human body includes a layer-shaped body that has a shape of surrounding the body between a wrist and a fingertip of a hand of the human body and forms at least two layers that are at least partially overlapped with each other, and at least one sensor that is provided on at least one of the at least two layers.

The present application claims priority from Japanese patent application JP 2022-78857 filed on May 12, 2022, the content of which is hereby incorporated by reference into this application.

### BACKGROUND OF THE INVENTION

### 1. Field of the Invention

The present invention relates to a wearable sensor including a sensor that measures an action related to fingers such as finger movement and a wearable system that collects and processes information measured by the wearable sensor.

### 2. Description of Related Art

Currently, there are many countries in the world where decline of birthrates and aging populations are progressing or expected to progress in the future. In Japan, the number of skilled workers is decreasing, for example, in the industrial field due to the effects of population decline and aging. Therefore, it is an urgent issue to pass on the skills from skilled workers to young workers. Many of the skills acquired by skilled workers require a long period of time to master. Therefore, it is required to measure actions of workers, quantify the actions, and use quantified data to enable workers to understand tips of the work. It is required to shorten the period required for the transfer of skills by the technique. Even in fields other than the industrial field, it is possible to measure actions performed by a human and use the measured actions to pass on skills.

There are various ways to measure the actions of workers, but one of the effective ways is to have workers wear sensors while working. Examples of the method of working while wearing sensors include the use of sensor gloves that can visualize the movements of the hands and fingertips, which are particularly likely to show the skill level of the worker by sensors.

For example, JP2016-125931A discloses a data glove including: a glove body; and a first distortion sensor and a second distortion sensor installed in a region corresponding to a vicinity of a metacarpophalangeal joint on at least a back side of one finger from among a first finger to a fifth finger of the glove body, in which the first distortion sensor detects the expansion and contraction in a proximal and distal direction of the region, and the second distortion sensor detects the expansion and contraction in a horizontal direction of the region.

### SUMMARY OF THE INVENTION

With the method described in JP2016-125931A, it is considered that the bending and stretching of fingers can be three-dimensionally and accurately understood. However, in manufacturing, for example, there are various movements in which workers can take actions. The items to be measured and the types of sensors to be used differ depending on the movements. Examples of items to be measured include bending and stretching of fingers, pressure applied to hands, acceleration thereof when the hands are moved, and sound generated during a work. Examples of the sensors to be used include a strain sensor, a pressure sensor, an acceleration sensor, and a microphone. For gloves that are specialized in using specific sensors, such as the data glove disclosed in JP2016-125931A, it is difficult to freely replace sensors to be used for changing items to be measured according to various actions of the workers.

An object is to provide a wearable sensor and a wearable sensor system in which sensor configurations can be easily rearranged so that a large number of actions related to fingers can be measured in various fields, including the industrial field.

A representative example of ways for solving the problems of the invention is as follows. That is, a wearable sensor to be worn on a human body is characterized by including a layer-shaped body that has a shape of surrounding a body between a wrist and a fingertip of the human body and forms at least two layers at least partially overlapping each other and at least one sensor provided on at least one of the at least two layers.

According to one aspect of the invention, the wearable sensor according to the invention can be used by combining two or more layers in an appropriate combination according to the application from a plurality of layers on which sensors are provided. Problems, configurations, and effects other than those described above are clarified by DESCRIPTION OF THE INVENTION below.

### BRIEF DESCRIPTION OF THE DRAWINGS

FIG. 1 is an example of an overall configuration diagram of a wearable sensor system;

FIG. 2 is a diagram illustrating a combination of gloves as a plurality of layers;

FIG. 3 is a diagram illustrating a configuration example of a sensor data collection unit;

FIG. 4 is a diagram illustrating an example of an operation screen for selecting a combination of gloves in the wearable sensor system;

FIG. 5 is a diagram illustrating a configuration example of a screen displaying measurement data of a wearable sensor in the wearable sensor system;

FIG. 6 is a diagram illustrating an example of a combination of wearable sensors as a plurality of layers; and

FIG. 7 is a diagram illustrating an example of storing a sensor in a pocket portion of a wearable sensor as a layer.

### DESCRIPTION OF THE INVENTION

A mode for carrying out the invention is described using the drawings as embodiments. Note that embodiments described below do not limit the invention according to the claims, and that all elements and combinations thereof described in the embodiments are not essential to the solution of the invention.

### Embodiment 1

By using wearable sensors to measure actions of workers, quantify the actions, and use quantified data, it is possible to enable workers to understand tips of the work. The period required for the transfer of skills by the technique can be shortened.

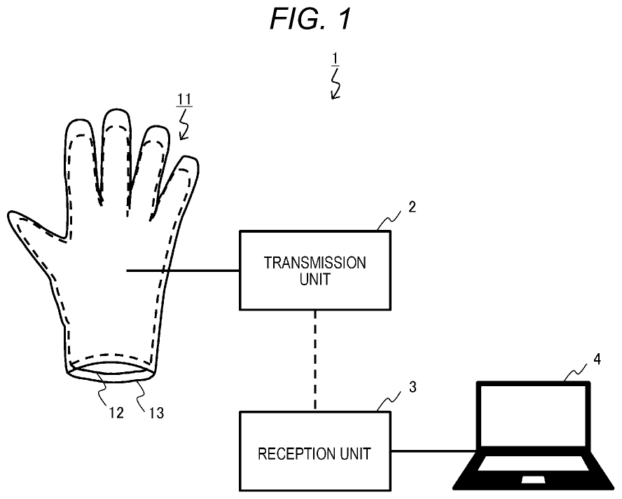

FIG. 1 is an example of an overall configuration diagram of a wearable sensor system. A wearable sensor system 1 includes a wearable sensor 11, a transmission unit 2, a reception unit 3, and a sensor data collection unit 4.

The wearable sensor according to Embodiment 1 includes a layer-shaped body surrounding a body between a wrist and a fingertip of the body, the layer-shaped body includes at least two layers at least partially overlapping each other, and at least one sensor may be provided on at least one of the at least two layers.

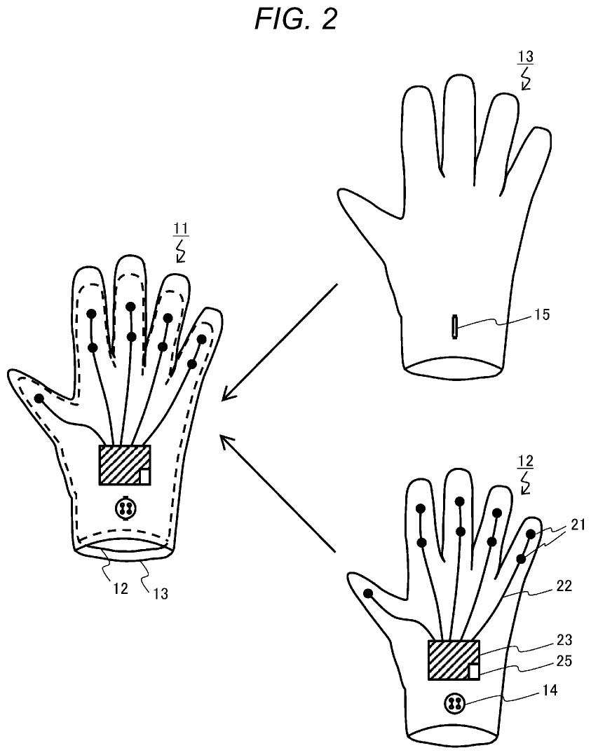

FIG. 2 illustrates a configuration example of the wearable sensor 11 according to Embodiment 1. Embodiment 1 is a wearable sensor having a glove shape. The wearable sensor having a glove shape may be simply referred to as a sensor glove or merely a glove. A case where the wearable sensor according to Embodiment 1 is used for a work mainly in the industrial field is described.

According to Embodiment 1, the functions of the wearable sensor can be easily changed by facilitating the change of the function of the wearable sensor. In Embodiment 1, a plurality of gloves including at least one sensor selected from a plurality of sensors can be prepared. The configuration of the wearable sensor can be easily changed by using a glove suitable for the work to be measured from among the plurality of gloves. It is also possible to use gloves without sensors together with gloves with sensors.

In other words, by configuring a wearable sensor by combining a plurality of gloves with different functions, the function of the wearable sensor can be changed by changing the combination of gloves. The wearable sensor 11 according to Embodiment 1 includes a layer-shaped body surrounding the body between the wrist and the fingertip of the body and includes an inner glove 12 and an outer glove 13 as at least two layers that are at least partially overlapped with each other.

The inner glove 12 includes sensors 21, a wiring 22, and a printed circuit board 23. There are at least one or more sensors in the inner glove 12, and a plurality of kinds of sensors may be used at the same time. In FIG. 2, with respect to the sensors 21, reference numerals are applied to only a part of sensors, but large black circles may indicate the sensors 21. With respect to the wiring 22, reference numerals are applied to only a part of wiring, but lines connecting the sensors 21 and the printed circuit board 23 are understood as the wiring 22. In FIG. 2, with respect to the wearable sensor 11, the sensors 21, the wiring 22, the printed circuit board 23, and a button 14 described below in the inner glove 12 are inside the outer glove but are drawn with solid lines to improve the visibility of the drawing. As for the material of the inner glove 12, various materials such as films, cloths, and leather materials made of natural or synthetic materials may be used. The inner glove 12 may be made by sewing or adhering cloth or may be made by molding a material by using a formwork.

Items to be measured and types of sensors to be used differ depending on an action of a person to be measured who works while wearing the wearable sensor, and thus it is desirable to select gloves with appropriate sensors according to the movement of the person to be measured. Examples of the items to be measured include bending and stretching of fingers, pressure applied to hands, acceleration when moving the hands, and work sound, and examples of the sensor to be used include a strain sensor, a pressure sensor, an acceleration sensor, and a microphone.

Here, in summary, the sensors may specifically be a pressure sensor, a strain sensor, an acceleration sensor, a gyro sensor, a geomagnetic sensor, a ranging sensor, a contact sensor, a temperature sensor, a Hall element, and a microphone. Table 1 shows an arrangement example for the inner layer and the outer layer of the at least two layers of the wearable sensor with respect to a sensor and a notification unit.

TABLE 1

| | Arrangement example of sensor | | |
|---|---|---|---|
| | and notification unit | | |
| Sensor/ | Sensor | | Reason for |
| notification | mounting | Inner layer/ | setting inner and |
| unit | point | outer layer | outer layers |
| Pressure | Palm side, | Inner layer | Emphasis on positional |
| sensor | fingertips | | relationship with fingers |
| Strain | Joint portion | Inner layer | Emphasis on shorter |
| sensor | | | distance to fingers |
| Microphone | Back of hand | Outer layer | Receive external sounds |
| | | | without blocking |
| Inertial | Back of hand | Inner layer/ | By structure |
| sensor | | outer layer | |
| LED | Back of hand | Outer layer | Easy to see |
| indicator | | | |

Here, a work is exemplified, and an example of sensors required to measure actions of a worker in that work is described.

One example of work is a painting. In the painting, a worker holds a spray gun and moves the spray gun left, right, up, and down while spraying paint. The moving speed of the spray gun is changed appropriately. Meanwhile, the work is done while controlling the amount of paint to be sprayed by a trigger. In training of the painting, it is necessary to transfer skills such as tips and techniques of skilled workers to learners, but it is difficult to transfer such skills by word. In applying the wearable sensor according to the invention to the painting, when the layer-shaped body having a glove shape is used as shown in the wearable sensor 11, the worker may wear the pressure sensor on a fingertip of a glove wore on a hand to operate the spray gun, in many cases, a portion corresponding to a fingertip of an index finger. A microphone as a sensor may be installed, for example, to the back of a hand of a glove corresponding to the hand that operates a spray gun. An inertial sensor may be installed to the portion of the back of the hand of the glove that corresponds to the hand that operates the spray gun. By arranging the sensors as such, the relationship between timing of pulling the trigger and force can be measured by the pressure sensor. The sprayed amount of the paint can be measured by the microphone in terms of sound. By the inertial sensor, the timing and speed of swinging the spray gun left and right and the like can be measured. By using measurement results when skilled workers do painting by using the wearable sensor according to the invention, learners can know how to operate spray guns by comparing the measurement results with results obtained by measurement on their own works.

Another work example is a connector installation work in network construction work or the like. Work mistakes such as mistakes in unplugging and plugging in connectors during a maintenance work in the field of information technology have a considerable impact, such as causing data loss. There are situations considering decrease of such work mistakes. In the connector installation work, a worker connects a male connector to a female connector. An example in which the wearable sensor according to the invention is applied to the connector installation work is described. As illustrated in the wearable sensor 11, when the layer-shaped body having a glove shape is used, sensors can be installed as follows. That is, the pressure sensors may be installed to fingertips of a glove that a worker wears on a hand holding a connector, in many cases, portions corresponding to fingertips of an index finger and a thumb. A microphone as a sensor may be installed, for example, to a portion of the back of the hand of the glove corresponding to the hand holding the connector. The inertial sensor may be attached a portion of the back of the hand of the glove corresponding to the hand holding the connector. By attaching the sensors as such, during the connector installation work, the amount of force for holding the connector can be measured by the pressure sensor, sound when the connector is fitted can be measured by the microphone, and the movement of the hand can be measured by the inertia sensor. By using measurement results when skilled workers work on connector installation by using the wearable sensor according to the invention, learners can know how connectors should be installed by comparing the measurement results with results obtained by measurement on their own works.

The description of the wearable sensor 11 is continued.

The wiring 22 may be wiring that connects the sensors 21 and the printed circuit board 23. The wiring 22 may be a cable and may be a part of the printed circuit board 23, and a printed circuit board different from the printed circuit board 23 may be used. In FIG. 2, each piece of wiring is represented by one line for the sake of simplification of the drawing, but such drawing is for the sake of convenience and does not limit the actual number of pieces of wiring. The wiring 22 may transmit the output of the sensor 21 to the printed circuit board. The wiring 22 may supply power required for the operation of the sensor from a battery 25.

The printed circuit board 23 is a printed circuit board that is electrically connected to the wiring 22, and data of a sensor may be collected on the printed circuit board 23. The printed circuit board 23 may be configured by a flexible circuit board, but is not limited to the printed circuit board, and may be any circuit board that integrates the outputs of the sensors. The battery 25 may be arranged on a part of the printed circuit board 23. The printed circuit board 23 may be equipped with a function of processing sensor data by calculation and a function of determining whether a work is to be performed and whether the work is good based on the sensor data. Here, what is simply called sensor data may mean data that is to be output or that is output by a sensor provided in the wearable sensor. Unless otherwise specified, the same is applied to the following description.

Power for the sensors 21, the transmission unit 2, and the like may be supplied from the battery 25 provided on the printed circuit board 23 or may be supplied from outside the glove. The sensors 21 and the transmission unit 2 may be configured that the power is supplied from each battery owned by the sensors 21 and the transmission unit 2.

The wearable sensor according to Embodiment 1 may include at least one notification unit in at least one layer among the at least two layers. The notification unit may notify the surroundings such as the worker wearing the wearable sensor. For example, the notification unit may include an LED display device, a vibration motor, and a sound generation unit such as a speaker for notifying the worker based on the data collected by the wearable sensor 11. Here, the LED display device, the vibration motor, and the sound generation unit are the notification units. When the worker is training or learning for work by using the wearable sensor 11, if a work action measured by the sensor installed in the wearable sensor 11 exceeds an allowable error in comparison with data as a reference, the LED display device, the vibration motor, and the sound generation unit may inform the worker of the fact. Alternatively, the notification may be given when the work motion measured by the sensor installed in the wearable sensor 11 is within the allowable error compared with the data as the reference. By providing such notification units, there is an effect that the worker using the wearable sensor can be notified of an awareness that leads to improvement of the work.

At least two layers of the wearable sensor according to Embodiment 1 may have different functions. For example, in addition to the electrical functions of sensing by the sensor and notification by the notification unit, the inner glove 12 may have a function of the material of the layer-shaped body as the function thereof. A case where the inner glove 12 as the layer-shaped body is formed by using a rubber material is exemplified. Since the rubber material is waterproof, a waterproof function to prevent the sensor from being damaged by the sweat of the worker can be provided.

The outer glove 13 may be a glove that is put over the outer side of the inner glove 12 for use. The outer glove 13 may be a glove having at least one function different from the inner glove 12. In addition to the electrical functions of sensing by the sensor and notification by the notification unit, the functions of the outer glove may include, for example, a cut-resistant function, a waterproof function, and a flameproof function. The function of protecting the inner glove 12 when the hand wearing the wearable sensor 11 comes into contact with an obstacle may be not provided to the inner glove 12 but provided only to the outer glove 13. As the material of the outer glove 13, various materials such as films, cloths, and leather materials made of natural or synthetic material may be used. The outer glove 13 may be made by sewing or adhering cloth or may be made by molding a material using a formwork.

In the wearable sensor according to Embodiment 1, the at least two layer-shaped bodies may be connected to each other by a stop member.

For example, the inner glove 12 and the outer glove 13 that are two layers can be fastened together by passing the button 14 through a buttonhole 15 and can also be removed. The button 14 may be a stop member of the inner glove 12, which is the inner layer-shaped body. The buttonhole 15 may be a stop member of the outer glove 13, which is the outer layer-shaped body. The button 14 and buttonhole 15 allow the inner glove 12 and the outer glove 13 to be easily fastened and removed.

The method of fastening the inner glove 12 and the outer glove 13 may be hooks, hook-and-loop fasteners, sewing, the use of adhesives, or the like. The position where the stop member is provided is not limited to the positions of the button 14 and the buttonhole 15 illustrated shown in FIG. 2, that is, the back side of the wrist of the glove. That is, for example, the fingertips of the inner glove 12 and the outer glove 13 may be fastened together, or wrist portions may be fastened together.

In the embodiments described above, an example in which the two gloves of the inner glove 12 and the outer glove 13 are used is described, but a structure in which three or more gloves are overlapped may also be used.

The layer-shaped body and the layers included in the layer-shaped body according to Embodiment 1 do not necessarily have to cover everything beyond the wrist like a glove. That is, for example, the wearable sensor may have a glove shape, but the fingertips may be opened so that the fingers are exposed when worn. The wearable sensor may surround a wrist like a bracelet, may surround a finger like a ring, or may surround a fingertip like a finger sack. The wearable sensor according to the invention may be a layer-shaped body that surrounds the body, and at least a portion is overlapped. The upper and lower layers of the at least two layers, a layer on a side in contact with the body, that is, inside, and a layer opposite to the body, that is, outside, may not completely overlap with each other. That is, for example, the lower layer may have a glove shape, and the upper layer may have a finger sack shape, or the relationship between the upper and lower layers may be opposite.

In Embodiment 1, the inner glove 12 includes the sensors, and the outer glove 13 does not include the sensors, but contrarily, the outer glove 13 may include the sensors and the inner glove 12 may not include the sensors. Even in such a case, the essence of the invention does not change.

In the wearable sensor according to Embodiment 1, the at least two layers provided with the sensors may be connected to a transmission unit for transmitting output data of the sensors or a cable for data transmission. According to Embodiment 1, the wearable sensor 11 includes the transmission unit 2 in at least one of the inner glove 12 and the outer glove 13, and the transmission unit 2 may transmit the output data of the sensors in the at least one of the inner glove 12 and the outer glove 13. According to Embodiment 1, the wearable sensor 11 may be connected to a cable for data transmission that transmits output data of the sensors in the at least one of the inner glove 12 and the outer glove 13. By such a configuration, data measured by a sensor can be transmitted to the outside of the wearable sensor 11. As a result, data measured by the sensor can be used in various ways.

The wearable sensor system according to Embodiment 1 includes the wearable sensor according to the invention and the sensor data collection unit, the wearable sensor includes the transmission unit, the sensor data collection unit includes the reception unit, so that communication can be performed between the transmission unit and the reception unit.

FIG. 1 illustrates an example of the wearable sensor system according to Embodiment 1. In FIG. 1, the transmission unit 2 may be electrically connected to the printed circuit board 23 to transmit the sensor data received from the printed circuit board 23 or data obtained by processing the sensor data to the reception unit 3. The transmission unit 2 may correspond to a glove provided with a sensor among the plurality of gloves. The transmission unit 2 may be provided to each glove provided with sensors. As described below, the wearable sensor system may include one transmission unit 2 that collectively transmits output of sensors in the gloves with respect to the plurality of gloves provided with the sensors. Each of the sensors in the gloves may include the transmission unit 2.

A method of transmitting data from the transmission unit 2 to the reception unit 3 may be, for example, wireless connection such as connection by Bluetooth (registered trademark) or WiFi (registered trademark) or a wired connection using a cable or the like. The transmission unit 2 may have a function of processing the data transmitted from the sensor by calculation and a function of determining whether the work is to be performed and whether the work is good based on the sensor data. The transmission unit 2 may be integrated with the printed circuit board 23 and have a function of transmitting the sensor data to the reception unit 3 on the printed circuit board 23.

The reception unit 3 plays a role of receiving data transmitted from the transmission unit 2 and delivering the data to the sensor data collection unit 4. The reception unit 3 may have a transmission function in addition to a reception function, and the transmission unit 2 may have a reception function in addition to a transmission function. That is, the transmission unit 2 and the reception unit 3 may be a communication unit having a communication function capable of performing transmission and reception or may perform communication between communication units on both sides. As a result, for example, a function of lighting an LED provided to the printed circuit board 23 of the wearable sensor 11 and transmitting a signal to operate a vibration motor may be provided.

The sensor data collection unit 4 may perform various processes related to the received output data of the sensor. The sensor data collection unit 4 may include, for example, a personal computer (PC) having a processor, storage resources, and the like, a general-purpose computer, or a cloud server.

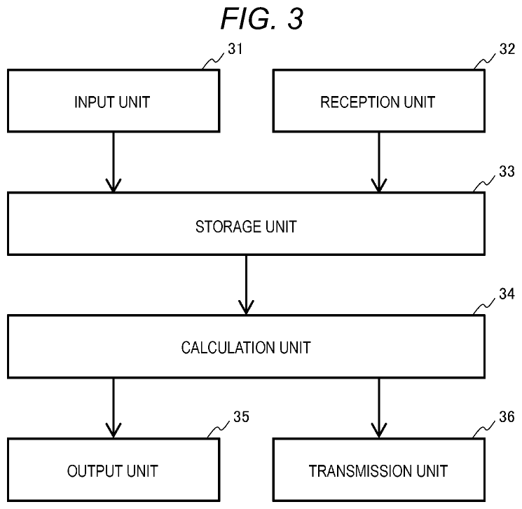

FIG. 3 is a diagram illustrating a configuration example of the sensor data collection unit 4. The sensor data collection unit 4 mainly includes an input unit 31, a reception unit 32 of the sensor data collection unit, a storage unit 33, a calculation unit 34, an output unit 35, and a transmission unit 36.

A function of processing the sensor data by the sensor data collection unit 4 may be a program stored in the storage resource. The sensor data collection unit 4 processes the output data of the sensor, generates a signal for performing the function of lighting an LED provided to the printed circuit board 23 of the wearable sensor 11 or the like described above, and transmits the generated signal. Here, the sensor data collection unit 4 may compare the work action measured by the sensor installed in the wearable sensor 11 with data as the reference, determine whether the work action exceeds the allowable error or is within the allowable error, and perform an LED lighting command.

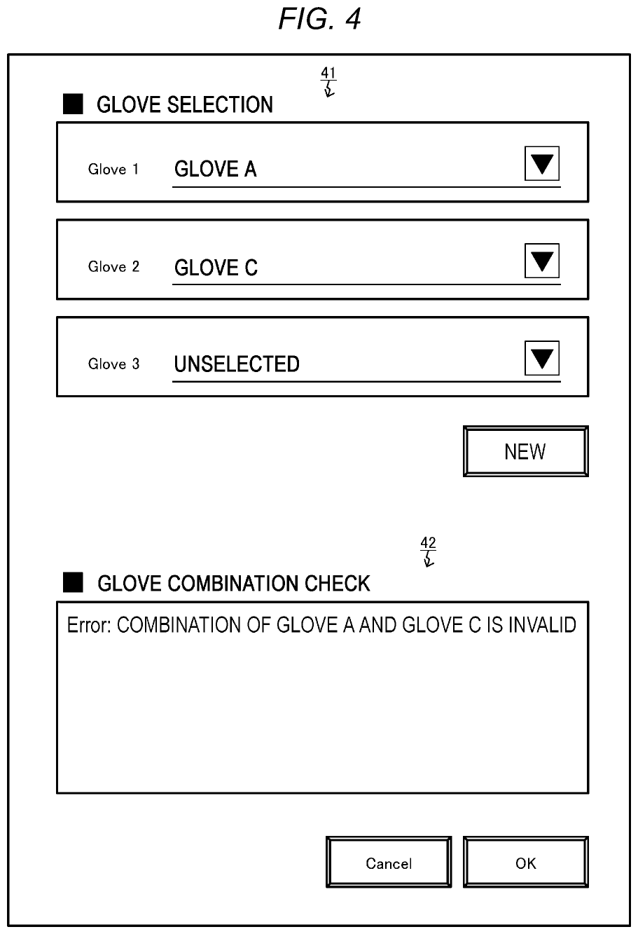

FIG. 4 is a diagram illustrating an example of an operation screen for selecting a combination of gloves in the sensor data collection unit 4 and illustrates an example of information input by the input unit 31. The operation screen may be displayed on a display. The display may function as the output unit 35 described below.

According to Embodiment 1, since the configuration of the wearable sensor can be easily changed by changing the combination of the gloves, items to be measured and types of sensors to be used can be appropriately selected according to the movement of the worker to be measured, that is, the work. Therefore, also in the sensor data collection unit 4, it is required to collect the data of the sensor according to the combination of the gloves. A glove selection area 41 is an area for selecting a glove registered in the system in advance by using a pull-down or the like. The number of gloves that can be selected is one or more and may be any number regardless of the example of the operation screen of FIG. 4. In the registration of the glove, a single glove before the combination of the plurality of gloves, for example, a glove that may be used as an inner glove or a glove that may be used as an outer glove may be registered. If there is a combination of gloves that is frequently used, the combination of gloves may be registered. The glove selection area 41 may have a function for registering a new glove and a new registration button for the function. The invention is related to a wearable sensor exemplified to have a glove shape, and the glove selection area 41 may be understood in an extended manner to have an area for selecting a wearable sensor different from the glove sensor or selecting a sensor. A glove combination confirmation area 42 is an area for displaying a result obtained by determining whether the combination of gloves selected in the glove selection area 41 is appropriate. Examples of a glove that is inappropriately combined include a combination of a glove for outer glove that is the same as the glove for the outer glove.

The reception unit 32 of the sensor data collection unit includes an interface for receiving data from the reception unit 3. According to the configuration of the wearable sensor system 1, the reception unit 3 may be integrated with the reception unit 32 of the sensor data collection unit.

The storage unit 33 may include storage resources such as a semiconductor memory, a flash memory, a hard disk drive (HDD), a solid state drive (SSD), a volatile type memory, and a non-volatile type memory and may store various items of information including data received from the input unit 31 and the reception unit 32 of the sensor data collection unit, a program to be executed by the calculation unit 34, or information to be used for sensor data collection and analysis.

The calculation unit 34 may include a processor and may perform various processes by executing the programs of the storage unit 33. For example, the calculation unit 34 may perform calculation on information related to the combination of the gloves received by the input unit 31, sensor data received from the reception unit 32 of the sensor data collection unit, or data obtained by processing the sensor data. The calculation unit 34 can perform calculation for graphically displaying the sensor data, calculation for determining whether the work is to be performed or whether the work is good, calculation for generation of a signal for lighting an LED provided to the printed circuit board 23 of the wearable sensor 11 or driving a vibration motor, and the like.

The output unit 35 may be able to display a graph, a determination result, or the like on the screen together with results of the calculation performed by the calculation unit 34. When the sensor data collection unit 4 is used in combination with another system, the sensor data collection unit 4 may output information delivered to the corresponding system.

The transmission unit 36 can transmit signals for lighting the LED provided to the printed circuit board 23 of the wearable sensor 11 and driving the vibration motor to the reception unit 3, for example, based on the calculation of the calculation unit 34. Here, the reception unit 3 transmits the transmitted signal to the transmission unit 2, and thus the transmission unit 2 and the reception unit 3 both may be transceivers having the transmission function and the reception function.

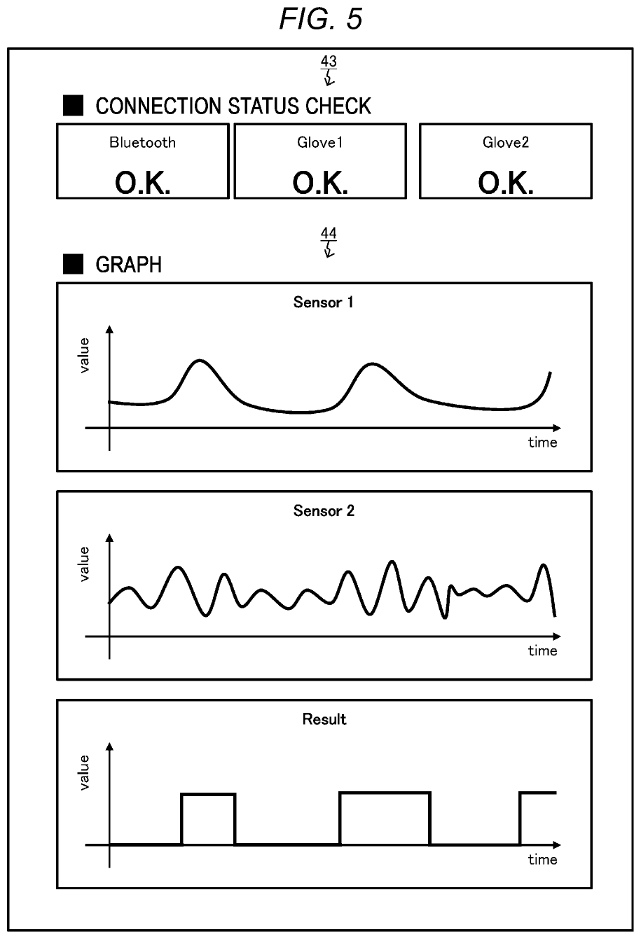

FIG. 5 is a diagram illustrating a configuration example of a screen that displays sensor data in the sensor data collection unit 4. The screen that displays the sensor data may display the sensor data updated in real time or may display sensor data that is stored. Here, the real time may mean that the time until the action of the worker who wears the wearable sensor becomes sensor data and is displayed on the sensor data collection unit 4 is a short period of time. The short period of time may be within 5 seconds, within 1 second, within 1 millisecond, and within 1 microsecond.

A connection status confirmation area 43 is an area for showing whether various connections are correctly performed and whether the sensor data is received to the sensor data collection unit 4. In the connection status confirmation area 43, it is displayed that the transmission unit 2 and the reception unit 3 are connected via Bluetooth (registered trademark), WiFi (registered trademark), and the like and that the glove is correctly connected. Here, various connections may be connection from the sensors installed to the glove, to the sensor data collection unit 4 via the transmission unit 2 and the reception unit 3. The checking whether the various connections are correct connections may be determined from the number of items of received data or may be determined from the feature of the sensor data. That is, checking can be determined based on whether the number of items of sensor data corresponding to the number of the sensors registered in advance are received or whether the sensor data corresponding to the feature of the sensor registered in advance is received.

The wearable sensor system according to Embodiment 1 may have a function of outputting sensing information of the sensor provided to the wearable sensor.

As illustrated in FIG. 5, the wearable sensor system according to Embodiment 1 may have, for example, a function of displaying or outputting sensor data that senses information of the sensor or data graphically showing a result obtained by processing the sensor data to a graph area 44. The displayed graph may display elapsed time of measurement on the horizontal axis and values obtained by converting the sensor data into an appropriate unit system on the vertical axis. The graph may show the result obtained by determining whether the work is to be performed or the work is good or may be a summary of the frequency of the work. The graph area 44 may have a mechanism for displaying a skill level of the worker or may show the movement of the hand of the worker by a figure imitating the wearable sensor. As such, by displaying the result obtained by measuring the work with the sensor with the graph, the worker who wears the wearable sensor can objectively understand the results of their own work. Three graphs exemplified in the graph area 44 may be displayed with aligned time axes, and the display of the graphs with aligned time axes enables the timing of the work action corresponding to each graph to be grasped easier. By using the graphs displayed in the graph area 44, data of a result of a work performed by a worker who trains the work and data of a result of the same work performed by a skilled worker may be able to be compared with each other and displayed. As such, tips of a work that are difficult to verbalize can be easily understood by vision by using the graphs, and thus the graphs help in communication when a trainee and the skilled worker make a conversation.

According to the wearable sensor according to the present embodiment, it is possible to use a combination of two or more layers that are appropriately combined according to the application from a plurality of layers on which sensors are provided. The combination makes it possible to easily prepare a wearable sensor suitable for the application and reduce the total number of types of wearable sensors to be prepared.

### Embodiment 2

In the wearable sensor according to Embodiment 2, sensors of at least two layers each including sensors may be electrically connected to each other between the two layers.

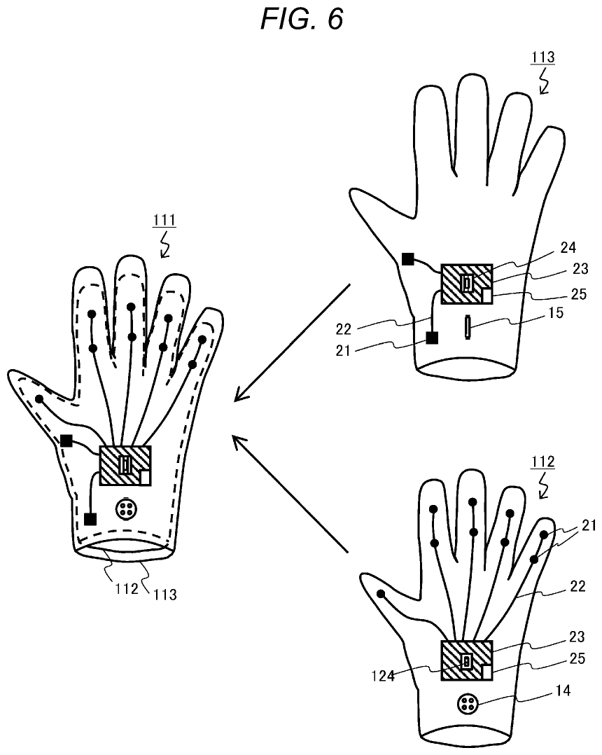

Embodiment 2 as a second embodiment of the invention is described with reference to FIG. 6. Embodiment 2 is in common with Embodiment 1 except for the points described below.

FIG. 6 is a diagram illustrating a configuration example of a wearable sensor 111 according to Embodiment 2 of the wearable sensor according to the invention. In Embodiment 2, in addition to an inner glove 112, an outer glove 113 may also include a sensor. The outer glove 113 may include the sensors 21, the wiring 22, and the printed circuit board 23. The sensors of the outer glove 113 are one or more types, and a plurality of types may be simultaneously used. The inner glove 112 and the outer glove 113 may respectively include connectors 124 and 24, for electrical connection. The connectors 124 and 24 may be a pair for combining connectors of the inner glove 112 and the outer glove 113. For example, the connector 124 of the inner glove 112 may be a female connector, and the connector of the outer glove 113 may be a male connector corresponding to the connector 124. By configuring the connectors as such, even if the inner and outer gloves are changed to gloves having different functions such as different provided sensors, the inner and outer gloves after change can be connected by the connector. The wearable sensor is not limited to two gloves of the inner glove and the outer glove and may be configured with three or more gloves. In a configuration of three or more gloves, for example, the innermost glove may include a female connector, and the gloves other than the innermost glove may include male connectors corresponding to the female connector. The male connector here may be able to connect three or more gloves by using a connector including a female connector on the back side where the connector pins are formed. The connectors of the inner glove 112 and the outer glove 113 each may be configured to be connected to the transmission unit 2. The sensors 21 of the inner glove 112 and the sensors 21 of the outer glove 113 are drawn with different figures, but the different figures simply distinguishes the sensors of the inner glove and the outer glove and are sensors with no difference.

In Embodiment 2, as in Embodiment 1, at least two layers where sensors are provided may be connected to a transmission unit that transmits output data of the sensor or a cable for data transmission. In Embodiment 2, the wearable sensor 111 includes the transmission unit 2 on at least one of the inner glove 112 and the outer glove 113, and the transmission unit 2 may transmit output data of the sensors of at least one of the inner glove 112 and the outer glove 113. In Embodiment 2, at least two gloves that are layers with sensors each may include the transmission unit 2 and each may be connected to cables for data transmission.

In the wearable sensor according to Embodiment 2, sensors of the at least two layer-shaped bodies to which sensors are provided may electrically connect the corresponding two layer-shaped bodies each other. In Embodiment 2, a combination of signals from the sensors of the inner glove 112 and a combination of signals from the sensors of the outer glove 113 may be connected between the inner glove 112 and the outer glove 113 by wireless communication. Each individual or group of sensors in each of the inner glove 112 and the outer glove 113 may be connected to other individual or group of sensors by wireless communication.

In Embodiment 2, the inner glove 112 and the outer glove 113 may be selected so that sensors are appropriately combined as the wearable sensor 111. Appropriately combined may mean that a glove including sensors that match the movement is selected, since an item to be measured or a type of a sensor to be used differs according to the movement of a worker to be measured, that is, a work. Examples of the items to be measured include bending and stretching of fingers, pressure applied to hands, acceleration when moving the hands, and work sound, and examples of the sensor to be used include a strain sensor, a pressure sensor, an acceleration sensor, and a microphone. For example, if the items to be measured are pressure and work sound, a glove with a pressure sensor may be selected as the inner glove 112, and a glove with a microphone may be selected as the outer glove 113. As such, by combining gloves with different sensors for use, it is possible to easily change the combination of sensors by combining gloves according to the application, and there is no need to create a dedicated wearable sensor for each application. That is, from a plurality of layer-shaped bodies provided with sensors, two or more layer-shaped bodies that are appropriately combined according to the application can be combined and used. A layer-shaped body appropriate for the application can be easily prepared, and types of layer-shaped bodies to be prepared, that is, a total number of wearable sensors can be reduced. As described above, some sensors are appropriate for arrangement on the inner glove, and some sensors are appropriate for arrangement on the outer glove. For example, the positional relationship with the fingers is important for a pressure sensor for measuring the pressure applied to the fingertip or the like, and thus it is effective to arrange the pressure sensor in the inner glove close to the fingers. Meanwhile, the microphone for recording work sounds should be arranged on the outer circumference side of the glove to avoid the sound being blocked by materials such as cloth of the glove as much as possible, and thus it is effective to arrange the microphone on the outer glove. As such, the glove prepared in advance has the effect of realizing a large number of combinations of sensors with a small number of types so that the arrangement can be considered according to the features of the sensors and more applications can be accommodated. Also, in Embodiment 2, functions of the inner glove 112 and the outer glove 113 are not limited to a function related to sensing, and for example, the inner glove 112 may have a waterproof function to protect the sensor from sweat of the worker, and the outer glove 113 may have functions of cut resistance, waterproofing, flameproofing, slip resistance, and the like.

The wearable sensor system according to Embodiment 2 may have a function of confirming that all combinations of layer-shaped bodies are valid combinations.

In the Embodiment 2, in the glove combination confirmation area 42, whether the combination of gloves used in combination and the combination of sensors are appropriate may be confirmable. For example, in the case of a combination in which the same sensors are arranged in the facing positions of the inner glove 112 and the outer glove 113, the user may be able to be notified that the combination of the gloves is inappropriate because of redundancy. If the connectors of the inner glove 112 and the outer glove 113 cannot be combined with each other, there may be notification that the combination between the gloves is inappropriate.

The wearable sensor system according to Embodiment 2 may have a function of confirming that a communication unit of sensors, a communication unit of a data collection unit, and all the layer-shaped bodies provided with the sensors are electrically connected appropriately.

In Embodiment 2, the connection status confirmation area 43 may have a function of confirming whether all gloves including the transmission unit 2, the reception unit 3, and the sensors are correctly connected. Here, both the transmission unit 2 and the reception unit 3 may be communication units having transmission and reception functions. The connection of the gloves may be a case where the inner glove 112 and the outer glove 113 are connected by connectors, and the outer glove 113 is connected to the transmission unit 2 or may be a case where the connectors of the inner glove 112 and the outer glove 113 each are connected to the transmission unit 2. As such, the function of confirming whether element of gloves or the like are correctly connected may be implemented by the following methods. That is, the correct connection may be determined based on the number of data received or may be determined based on the features of the sensor data. That is, the correct connection may be determined based on whether the number of items of sensor data corresponding to the number of sensors registered in advance is received or based on whether the sensor data corresponding to the features of the sensors registered in advance is received.

Embodiment 2 may be implemented together with Embodiment 1 described above.

### Embodiment 3

The wearable sensor according to Embodiment 3 may have a detachable member for attaching and detaching the sensor and the layer on at least one of the sensor and the layer.

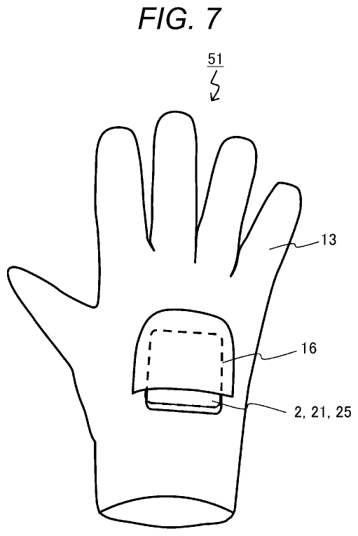

Embodiment 3 as a third embodiment of the invention is described with reference to FIG. 7. Embodiment 3 is in common with Embodiments 1 and 2 except for the points described below. Example 3 employs a glove as a layer.

FIG. 7 is a diagram illustrating Embodiment 3. Embodiment 3 may store a sensor in a pocket portion of a glove 51. In Embodiment 3, one glove is shown, but the one glove may be used as one of a plurality of gloves such as the inner glove and the outer glove in Embodiments 1 and 2. In Embodiment 3, at least one of a plurality of gloves that configure the glove 51 includes a pocket 16 as a detachable member that stores and detachably holds a sensor and wirings. The pocket 16 may have a structure that includes a cover with a hook-and-loop fastener, a button, or the like to prevent the sensor and the wiring from falling. Thus, even when the sensor and the wiring are stored in the pocket of the glove, the wearable sensor that obtains the same effects as those of Embodiment 1 or 2 can be configured. The sensor stored in the pocket 16 may be stored in the pocket 16 after being stored in the same housing as the transmission unit 2 and the battery 25. The sensors and the wirings stored in the pocket 16 may be electrically connected to other sensors or wirings, and the transmission unit 2 by the connector or the wiring, as required. The detachable member does not need to be provided only on the glove side as a layer. That is, the detachable member may be provided on the sensor side, the sensors and the glove may be attached and detached by the detachable member. The example may have a shape of a clip on which a sensor is provided. The sensor and the glove may each include a hook-and-loop fastener and can be attached and detached with the hook-and-loop fastener. By the configuration as in Embodiment 3, the sensor can be easily attached to and detached from the layer. As a result, many types of wearable sensors can be configured by preparing a small number of gloves, which are layers. Embodiment 3 as described above may be implemented together with Embodiments 1 and 2.

As described above, examples of embodiments of the invention are described using the first, second, and third embodiments, but the embodiments do not limit the invention according to the claims, and not all of the elements and combinations thereof described in the embodiments are essential to the solution of the invention. In the embodiments, the target of the measurement is described as a work. However, as long as an action has movement of hands or fingers such as sports or an action of playing a musical instrument, any action can be an object of the invention.

Although the invention is described in detail with reference to the accompanying drawings, the invention is not limited to such specific constructions but include various modifications and equivalent configurations within the spirit of the attached claims.

The invention is not limited to the above-described embodiments and can be embodied in the implementation stage by modifying the constituent elements within a range without departing from the gist of the present invention.

The invention is not limited to the embodiments described above and includes various modifications and equivalent configurations within the scope of the attached claims. For example, the embodiments described above have been described in detail for easier understanding of the present invention, and the invention is not necessarily limited to those having all the described configurations. A part of the configuration of one embodiment may be replaced with the configuration of another embodiment. The configuration of another embodiment may be added to the configuration of one embodiment. Additions, deletions, and replacements of other configurations may be made for a part of the configuration of each embodiment.

Each configuration, function, processing unit, processing means, and the like described above may be implemented by hardware, for example, by designing a part or all of them with an integrated circuit and or may be implemented by software by interpreting and executing programs that implement each function by a processor.

Information such as programs, tables, and files that implement each function can be stored in a storage device such as a memory, a hard disk, and a solid state drive (SSD), or a recording medium such as an IC card, an SD card, and a DVD.

As control lines and information lines, the lines that are considered necessary for explanation are shown, and not all the control lines and information lines necessary for implementation are necessarily shown. In practice, it may be considered that almost all configurations are connected to each other.

1. A wearable sensor that is worn on a human body, the sensor comprising:
a layer-shaped body that has a shape of surrounding the body between a wrist and a fingertip of a hand of the human body and forms at least two layers that are at least partially overlapped with each other; and
at least one sensor that is provided on at least one of the at least two layers.

2. The wearable sensor according to claim 1, wherein the at least two layers of the wearable sensor have different functions, respectively.

3. The wearable sensor according to claim 1, wherein the at least two layers are connected to each other by a stop member.

4. The wearable sensor according to claim 1, wherein the sensors are provided to the at least two layers, respectively.

5. The wearable sensor according to claim 1, wherein the sensor includes at least one of a pressure sensor, a strain sensor, an acceleration sensor, a gyro sensor, a geomagnetic sensor, a ranging sensor, a contact sensor, a temperature sensor, a Hall element, and a microphone.

6. The wearable sensor according to claim 1, further comprising at least one notification unit on at least one of the at least two layers.

7. The wearable sensor according to claim 4, wherein the at least two layers to which the sensors are provided are communicably connected to each other.

8. The wearable sensor according to claim 4, wherein the at least two layers to which the sensors are provided are connected to a first transmission unit that transmits a sensing result by the sensor, or a transmission cable.

9. The wearable sensor according to claim 1, further comprising a detachable member that detachably holds the sensor on the layer in at least one of the sensor and the at least two layers.

10. A wearable sensor system comprising:
the wearable sensor according to claim 1; and
a sensor data collection unit that collects an observation result from the wearable sensor, wherein
the wearable sensor includes a second transmission unit, and
the sensor data collection unit includes a reception unit that can communicate with the second transmission unit.

11. The wearable sensor system according to claim 10, wherein the sensor data collection unit has a function of confirming connection to a layer on which the second transmission unit and at least one of the sensors are provided.

12. The wearable sensor system according to claim 10, wherein the sensor data collection unit has a function of determining whether a combination of layers on which the sensors are provided.

13. The wearable sensor system according to claim 10, wherein the sensor data collection unit has a function of outputting a sensing result of the sensor provided to the wearable sensor.
